(19) United States
(12) Patent Application Publication
(10) Pub. No.: US 2013/0131009 A1
(43) Pub. Date: May 23, 2013

(54) Compositions Comprising High Molecular Weight Hyaluronic Acid and Methods For Producing Same

(71) Applicants: Vera Gorbunova, Honeoye Falls, NY (US); Andrei Seluanov, Honeoye Falls, NY (US); Christopher Hine, Brighton, MA (US)

(72) Inventors: Vera Gorbunova, Honeoye Falls, NY (US); Andrei Seluanov, Honeoye Falls, NY (US); Christopher Hine, Brighton, MA (US)

(73) Assignee: UNIVERSITY OF ROCHESTER, Rochester, NY (US)

(21) Appl. No.: 13/636,772

(22) PCT Filed: Mar. 24, 2011
(86) PCT No.: PCT/US11/29753

§ 371 Date: Feb. 4, 2013

Related U.S. Application Data
(60) Provisional application No. 61/317,136, filed on Mar. 24, 2010.

(51) Int. Cl. C12P 19/26

(52) U.S. Cl. CPC C12P 19/26
USPC 514/54; 536/53; 435/352; 435/84

(57) ABSTRACT

This invention provides cell culture compositions which produce significant quantities of high molecular weight hyaluronic acid. The cell cultures are obtained from cells of mole rats, such as naked mole rats and blind mole rats. The high molecular weight hyaluronic acid can be collected in the conditioned media of these cell cultures. These cell cultures provide a convenient source of large quantities of high molecular weight hyaluronic acid.

### CROSS REFERENCE TO RELATED APPLICATIONS

This application claims priority to U.S. Provisional Application No. 61/317,136 filed on Mar. 24, 2010, the disclosure of which are incorporated herein by reference.

### FIELD OF THE INVENTION

This invention relates to hyaluronic acid (HA) and more particularly to a high molecular weight HA and sources for producing large quantities of same.

### BACKGROUND OF THE INVENTION

HA is a non-sulfated glycosaminoglycan found in the extra cellular matrix of most cells, and increased amounts are found in connective, neural and epithelial tissues. Hyaluronic acid is made up of linear polymeric chains in which disaccharide units of N-acetylglucosamine and glucoronic acid, bonded via by glucoside bonds, are repeated. It has been reported to have roles in promoting contact inhibition through binding to the cell surface glycoprotein CD44. HA is widely used in supporting joint function in arthritis patients (such as via knee injections), beauty products, and veterinary medicine (knee injections for race horses). The currently commercially available HA is purified from bacteria or rooster combs. The cost of HA is 1-10 million dollars per kilogram, depending on the polymer length (the longer, more valuable). Currently used purification processes yield short polymers of inferior quality.

### SUMMARY OF THE INVENTION

The present invention provides compositions and methods for obtaining high molecular weight hyaluronic acid from animal cell cultures. Accordingly, in one aspect the invention provides cell culture compositions obtained from subterranean rodents such as mole rats. In one embodiment, the mole rats may be naked mole rats or blind mole rats. While any cells that can be propagated in culture can be used from these animals, it is convenient to obtain skin fibroblasts and maintain those in culture. Accordingly, in one embodiment, the cell culture compositions comprise primary, secondary or any passage cells or stably transformed cells obtained from skin fibroblasts of naked mole rats. In one embodiment, the cultured cells or fresh fibroblasts obtained from the animals are frozen and stored for later use.

In another aspect, the present invention provides methods for obtaining cell cultures from subterranean rodents such as mole rats including, but not limited to naked mole rats or blind mole rats. The method comprises obtaining cells that can be propagated in culture, such as fibroblasts. Thus, in one embodiment, fibroblasts are obtained from the skin of naked mole rats and cultured for several passages such that they spontaneously transform and no longer exhibit early contact inhibition. Cells that no longer exhibit early contact inhibition can also be obtained by transfecting the cells with suitable vectors such as SV40 large T antigen.

The present invention also provides conditioned medium from the cell cultures obtained from the cells (such as fibroblast cultures) of subterranean rodents such as mole rats including, but not limited to naked mole rats or blind mole rats. The conditioned medium can be collected any time after plating of the cells. The conditioned medium is rich in high molecular weight hyaluronic acid having a molecular weight of 6,000 kDa or more.

In another aspect, the present invention provides high molecular weight hyaluronic acid having a molecular weight of 6,000 kDa or more obtained from the conditioned medium of cultured cells from subterranean rodents such as mole rats including, but not limited to naked mole rats or blind mole rats.

### BRIEF DESCRIPTION OF THE DRAWINGS

FIG. 1. Increased viscosity of NMR media is not due to secreted proteins. (A) Proteins from unused cell culture media or conditioned media from NMRSF cells, which produce viscous media, or Naked mole rat skin fibroblast (NMREF) cells, which do not produce viscous media, were separated by SD-PAGE and detected by coomassie staining There were no detectable differences in aberrant protein expression between the three media types. This suggests that proteins are not the cause of increased viscosity of NMRSF media. The intense staining band in all three samples is due to bovine serum albumin (BSA) in the media.

FIG. 2 (A-D). Naked Mole Rat Cells Secrete High Molecular Weight Hyaluronic Acid by Upregulation of HAS2. (A) Relative viscosities of cell culture media, either unused or day 20 conditioned media from various cell lines. Naked mole rat skin fibroblast media (NMRSF) displayed increased viscosity over human skin fibroblast (HSF) and mouse skin fibroblast (MSF) media. This increased viscosity was abolished with the addition of hyaluronidase (HAse). (B) Hyaluronic acid (HA) abundance in day 20 NMR media from two different naked mole rats (NMRSF3 and NMRSF4), determined by equimolar competitive HA binding ELISA, was found to be relatively equal to that found in MSF cells and slightly increased compared to HSF cells. (C) Pulse field electrophoresis gel stained with Stains-All to determine the MW of HA secreted into the media by various cell lines. Removal of staining with the addition of HAse confirms HMW-HA expression in NMRSF and NMRSF Mutant (Mut) cells. (D). The NMRSF Mut. Cells are spontaneously transformed cells. However, no significant HMW-HA was observed in naked mole rat embryonic skin fibroblasts (NMREF). Western blot analysis of hyaluronic acid synthases (HAS) in HMW-HA expressing NMRSF cells and LMW-HA expressing NMREF.

FIG. 3 (A-C). HMW-HA promotes E.C.I. in NMRSF cells. (A and B) NMRSF cells grown in standard Eagle's Modified Essential Medium (EMEM) media display arrest by day 20 at low cell densities, which we termed early contact inhibition (E.C.I.). Removal of HMW-HA by HAse allows NMRSF cells to surpass E.C.I. and arrest at higher cell densities at complete confluence (C.C.). (C) NMRSF cells at C.C. due to the addition of HAse revert back to E.C.I. after the removal of HAse, thus permitting NMSF cells to continue production of HMW-HA.

FIG. 4. Cells from subterranean blind mole rats express more HMW-HA than NMR Cells. (A) Similar to FIG. 2C, media samples from day 20 NMRSF and day 7 blind mole rat were run on a pulse field agarose gel and stained with Stains-All to determine relative sizes of secreted soluble HA. Day 7 was chosen due to these cells from blind mole rats undergoing cell death at extended periods on the plate. Even within 7 days of growth, blind mole rat cells express what appears to be more and larger HMW-HA than do NMRSF cells. DNA ladders where used for comparison of relative sizes.

### DETAILED DESCRIPTION OF THE INVENTION

The present invention is based on the unexpected fining that cells cultured from mole rats produce significant amounts of high molecule weight (HMW) hyaluronic acid (HA), which can be conveniently collected in the conditioned medium obtained of these cells.

The cells can be obtained from any mole rat. For example, mole rats belonging to family Bathyergidea, family Spalacidae, or family Geomyidae can be used. Any mole rat species from these families can be used. For example, useful species from the Bathyergidea family include Naked Mole Rat (Heterocephalus glaber), Cape Dune Mole Rat (Bathyergus suillus), Namaqua Dune Mole Rat (Bathyergus janetta), Slivery Blesmol (Heliophobius argenteocinereus), Ghana Blemol (Fukomys zechi), Malawian Blesmol (Fukomys whytei), Ochre Blemol (Fukomys ochraceocinereus), Kataba Blesmol (Fukomys micklemi), Mechow's Blesmol (Fuomys mechowii), Kafue Blesmol (Fuomys kafuensis), Nigerian Blesmol (Fukomys foxi), Mashona Blesmol (Fukomys darlingi), Damaraland Blesmol (Fukomys damarensis), Bocage's Blesmol (Fukomys bocagei), Ansell's Blesmol (Fukomys anselli), Zambian Blesmol (Fukomys amatus), Matabeleland Mole Rat (Cryptomys nimrodi), Cape Blesmol (Georychus capensis). Examples of useful species from the Spalacidae family include Middle East Blind Mole Rat (Spalax ehrenbergi) (Superspecies), Sandy Mole Rat (Spalax aenarius), Mt. Carmel Blind Mole Rat (Spalax carmeli), Upper Galilee Mountains Blind Mole Rat (Spalax galili), Giant Mole Rat (Spalax giganteus), Golan Heights Blind Mole Rat (Spalaz golani), Balkan Mole Rat (Spalax graecus), Judean Mountains Blind Mole Rat (Spalax judaei), Lesser Mole Rat (Spalax graecus), Greater Mole Rat (Spalax microphthalmus), Munzur Mole Rat (Spalax munzuri), Nehring's Blind Mole Rat (Spalax nehringi), Kazakhstan Bline Mole Rat (Spalax auralensis), Podolsk Mole Rat (Spalaz zemni). Examples of useful species from Geomyidae family include Pocket gophers (Geomys, Heterogeomys, Orthogeomys, Pappogeomys, Thomomys, and Zygogeomys).

It was observed that embryonic cells obtained from these animals did not produce such high amounts of HMW HA. Accordingly, in one embodiment, non-embryonic cells are obtained from the mole rat. For example, cells can be obtained from adult mole rats. While any cells can be obtained and cultured from the mole rats, it is convenient to obtain fibroblasts (such as skin fibroblasts) because these can be conveniently maintained in culture.

In one embodiment, the mole rat is the naked mole rat (NMR) (Heterocephalus glaber). It is known for eusocial colony structure and behavioral characteristics. It is a small rodent with exceptionally long lifespan (up to 30 years) and resistance to cancer. We observed that cells obtained from the NMR are unable to form robust anchorage-independent growth when transfected with oncogenic Ras and SV40 Large T, while this combination readily transforms mouse fibroblasts. We have found that this resistance to oncogenic transformation is possibly due to NMR fibroblasts displaying hypersensitivity to contact inhibition, a phenomenon we termed “early contact inhibition” (“ECI”; also referred to herein as “E.C.I.”), as their fibroblasts arrest at a much lower density than those from a mouse or human. This early contact inhibition requires the activity of p53 and pRb tumor suppressor pathways and is associated with the induction of p16^{Ink4a}. We have determined upstream signaling of early contact inhibition is induced by NMR cells producing an excess of high molecular weight (>6,000 kDa) hyaluronic acid (HA).

NMR cells that undergo early contact inhibition overexpress high molecular weight HA, its synthase HAS2 and also CD44 when compared to early contact inhibition null embryonic NMR cells. This HMW-HA may then stimulate the ECI phenotype through interaction with an NMR specific isoforms or posttranslational modification of CD44 and results in maintaining the tumor suppressor NF2 in a growth prohibitive/anti-cancer dephosphorylated form. Removal of HMW-HA from NMRSF cultures resulted in the loss of ECI, the phosphorylation of NF2 and the ability of NMRSF cells to be oncogenically transformed and form robust colony growth in an anchorage independent soft agar assay. Thus, it appears loss of contact inhibition is necessary, but not sufficient, for oncogenic transformation of NMR fibroblasts. By removing HA from the media/extracellular matrix of NMR cells using hyaluronidase, we were able to grow NMR cells to complete confluence similar to human and mouse cultures. Furthermore, we could rescue the early contact inhibition phenotype of these fully confluent cells by removing hyaluronidase from the media. These results indicate the tumor suppressive mechanism of early contact inhibition in NMR cells is induced by high molecular weight HA. Due to HA's roles in extracellular matrix health, aging and anti-oxidant properties, the data may also explain why NMRs are unusually long lived. Our lab has identified several anti-cancer/anti-aging mechanisms in the NMR using a comparative biology approach. In the present invention, we have identified HA as the key mediator of early contact inhibition.

NMR skin, heart, brain and kidney tissues showed an abundance of HA, compared to other rodents. The HMW-HA secreted by NMR cells appears to offer resistance to both internal and external oxidative stress. We also showed this NMRSF derived HMW-HA was effective at providing resistance to oxidative stress to human cells. Due to the ease of harvesting culture media from NMRSF cells and obtaining HMW-HA in solution, these cells can be used for the production of highly desirable and clinically effective HMW-HA.

Accordingly, in one aspect, the invention provides a high molecular weight HA such that the molecular weight of HA is greater than 6,000 kDa as determined on agarose gel electrophoresis. In various embodiments, the molecular weight of HA is greater than 7,000, 8,000, 9,000 and 10,000 kDa (including molecular weight in kDa of all integers between 6,000 and 10,000). Thus, when a reference is made to a high molecular weight hyaluronic acid, it means HA which has a molecular weight of at least 6,000 kDa and in one embodiment, from 6,000 kDa to 10,000 kDa.

The high molecular weight HA can be obtained from the cells of any mole rat, such as a naked mole rat or a blind mole rat. A convenient cell type as a source of hyaluronic acid from these mole rats is fibroblasts. The fibroblasts can be obtained from mole rats of any age by techniques well known in the art. Generally, skin fibroblasts are easily obtained and methods for obtaining skin fibroblasts are well known in the art.

In one embodiment, primary fibroblasts or cell lines established from the primary fibroblasts can be used. For example, primary fibroblasts can be cultured from the skin of mole rats. Skin is obtained from mole rats by standard process. A convenient area for obtaining skin is the underarm area as the skin. Generally, the naked mole rats do not have fur, but if fur is present, it can be shaved first. The skin sample is then cut into fragments (such as 1 cm²). The skin fragments are further cut into smaller fragments (such as about 1 mm pieces). The fragments are then exposed to protease mixture to loosen the cells (such as Liberase Blendzyme 3, and 1×antibiotic/antimycotic) generally in a tissue culture medium (such as DMEM/F12). Digestion can be carried out at 37C for a desirable period (such as 30-90 minutes). Digestion can be stopped by removing the digestion medium or by adding enzyme inhibitors. Released cells can be collected, centrifuged and re-suspended in a suitable medium (such as DMEM/F12 with 10-15% FBS). The primary fibroblast cultures can be incubated at 37° C., 5% CO₂ ad 3% O₂). Conditioned medium can be obtained after suitable incubation times (such as 3 hours to 30 days and all times therebetween) and HA can be purified from the conditioned media to the extent desired or the conditioned medium can be used as such.

HA can be purified from the conditioned media by, for example, the following process comprising treating the conditioned medium with proteases, ethanol precipitation, resuspension of the precipitate in buffer and gel electrophoresis wherein HA can be identified by doing treatment with hyaluronidase.

We observed that primary naked mole-rat cell isolates produce high molecular weight HA but proliferate slowly. To simplify cell culture process and to make large quantities of HA-producing cells, in one embodiment, we obtained fast proliferating transformed naked mole-rat cell lines. Transformed cell lines can be obtained in at least two ways: (1) spontaneous transformation is sometimes observed when the primary cells are cultured for over 100 days; (2) by stably transfecting primary cells with SV40 large T antigen (Simian Vacuolating Virus 40 Tag; “LT”). LT is derived from polyomavirus SV40. Its amino acid sequence and nucleotide sequences encoding it are known in the art. It is a hexamer protein that can transform a variety of cell types. The mechanism by which it can be used to transform cells is known (see, for example, Ali S H, DeCaprio J A. Cellular transformation by SV40 large T antigen: interaction with host proteins. Semin Cancer Biol. 2001 February; 11(1):15-23) and relates to inactivation of two major and well characterized tumor suppressor pathways, Rb and p53. Polynucleotide vectors encoding LT that can be used to transform cells according to the method of the invention are available to the public from, for example, Addgene, Cambridge, Mass., USA, but the invention includes transforming cells using any suitable polynucleotide that encodes LT, or any derivative of LT that can transform eukaryotic cells. We have successfully produced several exemplary cell lines by transformation using LT-encoding expression vectors (plasmid 13970 from Addgene, Inc.). Thus, in another embodiment, the present invention provides stably transformed NMR cell lines which produce HA, particularly high molecular weigh HA.

Thus, in another embodiment, the present invention provides stably transformed NMR cell lines which produce HA, particularly high molecular weigh HA of the present invention. In one embodiment, the cells can be stably transfected. The terms “stably transfected”, as used herein in conjuction with cells obtained from mole rat mean cells into which has/have been introduced polynucleotide(s) encoding for SV40 large T antigen (LT), and which cells can be stably maintained and proliferated in culture, irrespective of whether or not the introduced polynucleotide has integrated into the genome of the cell. Thus, the cells may be genetically modified, epigeneticaly modified, or modified in some other way.

For the naked mole rat cells lines, we have obtained three independent lines that have lost the early contact inhibition phenotype (alternative spicing/expression of the CD44 protein) and these cells thus grow to higher cell density on the plate (easier to grow) and express hyaluronic acid similar to the wildtype lines. By the term “wildtype” is meant early passage fibroblast cells, which have not lost early contact inhibition. The spontaneously transformed (also referred to as stably transformed) cell lines have lost the early contact inhibition due to continuous passaging over a long period of time (such as for 100 days or more). Both the stably transformed and the wildtype cells produce the same type of HMW-HA.

Generally, the early passage cells (before 15 population doublings) exhibit early contact inhibition. After this, and generally observed at population doubling 20 (generally referred to as passage 20), the cells lose the early contact inhibition phenotype and can be deemed stably transformed.

We have also tested SV40 Large T expression in these lines transiently and they loose the early contact inhibition phenotype. The conditioned medium from these cells lines remains viscous. Additionally, we have also obtained fibroblasts from another species, the blind mole rat, and find the cells to have the same characteristics as the cells from NMR. The blind mole rat cells grow to complete confluence on the plate but have difficulty growing after several passages. However, stable integration of a construct expression SV40 Large T allows these cells to continue to grow. These cells, just as the wildtype blind mole rat cells, express the high molecular weight hyaluronic acid.

### Cell Culture

The present invention also provides compositions comprising cultured cells, such primary, secondary or any passage cells as well as long-term cultures of mole rat cells or from stably transformed mole rat cells such as fibroblasts. The mole rat cells can be cultured in monolayer, beads (i.e., two-dimensions) or in three-dimensional culture systems. The cells can be cultured by standard methods using aseptic processing and handling. The cells are cultured in the desired culture medium. In various embodiments the cells can be cells from the NMR or the BMR.

The cell culture medium in which the NMR cells are cultured can be any standard cell culture medium which provides adequate nutrition to the cells. Before being conditioned (such as before being added to the cells), the medium is termed as “pre-conditioned medium”. Suitable cell media include, but are not limited to EMEM, Dulbecco's Modified Eagle's Medium (DMEM), Ham's F12, RPMI 1640, Iscove's, McCoy's and other media formulations readily apparent to those skilled in the art. Such media can be easily prepared or obtained from commercial sources. Details of cell culture media and methods can be found in Methods For Preparation of Media, Supplements and Substrate For Serum-Free Animal Cell Culture Alan R. Liss, New York (1984) and Cell &Tissue Culture: Laboratory Procedures, John Wiley & Sons Ltd., Chichester, England 1996. The medium may be supplemented, with components, such as vitamins, growth factors, proteins, sugars, anti-oxidants, etc. as necessary to support the desired cell culture. Additionally serum, such as bovine serum, which is a complex solution of albumins, globulins and growth factors may be added if desired. For human use, if desired, animal serum be avoided. Instead, serum-free medium can be used or human plasma can be added in the same amount as animal serum. Hormones, growth factors or other agents may be added into the medium. The NMR cells generally take about 7 days to undergo one population doubling and therefore, over 100 days (about 140 days) are generally observed for 20 population doublings. After the cells are transformed, they can be passaged for as long as desired. This allows for a large scale up of NMR cells and therefore an unlimited source of HMW-HA.

The cells can be used in continuous cultures or can be frozen for later use. Techniques for freezing and reculturing frozen cells are well known in the art.

### Conditioned Media

The conditioned media obtained from the cells should be processed under sterile conditions or sterilized as needed. When appropriate (i.e., once the medium is conditioned so that hyaluronic acid or some other marker such as growth factors have reached desirable levels in the medium) the “conditioned” medium can be collected. In one embodiment, the conditioned media can be collected anytime after plating such as from 3 hours to 30 days of conditioning. In one embodiment, the conditioned medium is collected after 2, 3, 6, 12, 18, 24, 30, 36, 42, 48 hours, 3, 4, 5, 10, 15, 20, 25 or 30 days and all days and hours therebetween, and all ranges of time between 2 hours and 30 days. In one embodiment, it is collected anytime between 3 hours and 60 hours of incubation.

The mole rat cell derived conditioned medium is unique in that it contains a high level of high molecular weight hyaluronic acid. Therefore in one embodiment, this invention provides a neat (unprocessed) conditioned medium from the cells provided herein or partially or fully purified fractions of the medium comprising high molecular weight hyaluronic acid. In one embodiment, the conditioned medium from the cells comprises at least 50 ng/ml of HA having a molecular weight of at least 6,000 kDa. In various embodiments, the conditioned medium has at least 50 ng to 5 mg/ml of HMW-HA and all integer values and ranges in nanograms therebetween. In various embodiments, one ml of the unprocessed conditioned medium has at least 50, 100, 200, 300, 400, 500, 600, 700, 800, 900 ng, 1, 1.5, 2, 2.5, 3, 3.5, 4, 4.5, 5 μg of HMW-HA. In another embodiment, one ml of conditioned medium has 1 to 5 μg of HMW-HA.

If desired, the conditioned medium can be processed to concentrate selected components. For example, the medium may be concentrated 10 to 20 fold using a positive pressure concentration device (such as a device having a filter with a 0.2 or 0.45 μm cut-off (Amicon, Beverly, Mass.). Also, the conditioned medium may be further processed for HA isolation and purification to remove unwanted proteases. Methods of purification include ethanol precipitation followed by suspension in desired buffers including PBS, HEPES, TRIS and the like; gel chromatography, ion exchange, affinity chromatography HPLC purification and the like.

In one embodiment, the conditioned medium from the long term cultured or stably transformed fibroblasts can be collected once the cells are over 50% confluent. In various embodiments, conditioned medium can be collected when the cells are between 50 to 100% (and all integers therebetween) confluent. In one embodiment, the cells are 80-90% confluent. Once collected, the conditioned medium can be filtered to remove debris. As discussed above, the filtered medium can be used neat or can be processed to remove and/or concentrate desired components. The conditioned medium with or without processing can be used fresh or can be stored (at refrigerator or freezer tempearatures) for later use.

Accordingly, in one aspect of the invention, the invention provides conditioned medium that has previously supported the growth of long term cultured or stably transformed mole rat fibroblasts, such as NMR or BMR fibroblasts.

The HA of the present invention can be used in various pharmaceutical, neutraceutical, therapeutic or any other type of formulation that will come in contact with an individual or will be administered to an individual via any route of administration. Examples include but are not limited to medications for injection in arthritic joints, veterinary medicine, dietary supplement, and in cosmetics in creams and anti-wrinkle injections.

Thus, in one aspect, the present invention provides formulations comprising, consisting essentially of, or consisting of HA of the present invention.

The present invention also provides a method of alleviating a medical condition (which can be alleviated by administration of high molecular weight HA) by administration of HA containing formulations of the present invention.

### Example 1

### Materials and Methods

Animals.

Young naked mole rats (˜three years of age) were used for cell isolation and tissue analysis. Blind mole rats were also used for cell isolation and studies.

Cell Isolation and Culture.

All cell lines, once log phase growth was established after isolation, were grown on treated polystyrene culture dishes (Corning), unless noted, in EMEM media (American Type Culture Collection (ATCC)) supplemented with 15% FBS, nonessential amino acids, sodium pyruvate, 100 U/mL penicillin and 100 U/mL streptomycin (all supplements from Gibco). Human, mouse, and blind mole rat cells were grown at 37° C., 5% CO₂, 3% O₂ and 90% humidity while naked mole rat cells were grown at 32° C., 5% CO₂, 3% O₂ and 90% humidity. Human skin fibroblasts (HSF) were immortalized with hTERT (HCA2T). Old human skin fibroblasts (HSF Sen.) were obtained from the National Institute on Aging's Baltimore Longitudinal Study of Aging. Primary rodent skin fibroblasts were obtained as follows Skin fibroblasts were isolated from the underarm skin or embryonic tissues of NMRs, Mice, and Blind mole rats Skin was cleaned with 70% ethanol, shaved and tissue cut up and incubated in DMEM F-12 (Gibco) with 0.14 U/mL Liberase Blendzyme 3 (Roche) for 30-90 minutes to dissolve cells. Cells were then washed and placed on treated polystyrene tissue culture plates (Corning) with DMEM F-12 Media with 15% FBS and antibiotics/antimycotics (all from Gibco). Cells were incubated as described above until log phase growth and confluence were obtained. At this point, cells were transferred to the standard EMEM media described.

Viscosity Assay.

To determine relative kinematic viscosity, 3 mL of distilled H₂O, unused complete EMEM and harvested and day 20 conditioned media from naked mole rat skin fibroblasts; NMRSF, immortalized human skin fibroblasts; HSF, senescent human skin fibroblasts; HSF Sen, mouse skin fibroblasts; MSF, or NMRSF day 20 media treated with hyaluronidase; 1 U/mL hyaluronidase from Streptomyces hyalurolyticus (Sigma-Aldrich), were run through a 0.6 mm capillary Ostwald viscometer (Barnstead International) at 22° C. and timed for the passage of the media or ddH₂O through the capillary. Relative viscosities of unused and conditioned media was determined by comparing times required to pass through the capillary to that of ddH₂O, Samples were run three times to determine an average relative viscosity.

Hyaluronic Acid Concentration Determination.

Unused complete EMEM media, day 20 conditioned media from two different NMRSF, MSF and HSF cells lines were used to determine the relative hyaluronic acid concentration in the conditioned media by applying these samples to a hyaluronic acid enzyme-linked immosorbent assay (ELISA) kit (Echelon Biosciences). This HA-ELISA is a competitive binding assay where the colorimetric signal is inversely proportional to the amount of HA present in the sample. The assay was completed by following the manufacturer's guidelines. Briefly, 100 μL of HA standards, between 0 and 1200 ng/mL, and 100 μL of conditioned media samples were added to the wells of the incubation plate, followed by the addition of 50 μL of working detector to each well and incubated for one hour at 37° C. Next, the samples and controls were transferred to the HA ELISA plate and incubated for 30 minutes at 4° C., followed by discarding the samples and standards from the plate and washing with wash buffer. One hundred μL of the enzyme was then added to each well and incubated at 37° C. for 30 minutes and then washed again. One hundred μL of substrate solution was then added to each well and incubated in the dark at room temperature for 45 minutes. Samples and standards were read three times at 10-minute increments on a spectrophotometer at 405 nm. Concentrations of samples were determined by calculating the % binding for each standard and sample.

Hyaluronic Acid Pulse Field Gel.

Hyaluronic acid sizes in Day 20 conditioned media from human skin fibroblasts (HSF), mouse skin fibroblasts (MSF), naked mole rat fibroblasts (NMRSF), mutant naked mole rat fibroblasts that have lost E.C.I. by spontaneous transformation (NMRSF Mut.), embryonic naked mole rat fibroblasts (NMREF) and blind mole rat skin fibroblasts (BMRSF Day 7) were determined using pulse field gel electrophoresis. First, HA was concentrated from conditioned media samples by first treating 2 mL of conditioned media with 500 μg of Proteinase K (Roche) at 50° C. for 45 minutes to remove proteins. Samples were then precipitated by adding 2 mL of 100% ethanol, centrifuging for 5 minutes at 1500×g and followed by discarding the supernatant. The pellet was then dissolved in 500 μL TE Buffer and incubated overnight at 4° C. The following day, aliquots were removed and treated with hyaluronidase from Streptomyces hyalurolyticus (Sigma-Aldrich). Twenty-five μL of each sample that was treated plus/minus hyaluronidase and mixed with 5 μL 4M sucrose loading solution was loaded to a 0.4% pulse field SeaKem Gold agarose gel (Cambrex).10 μL of hyaluronic acid molecular size markers; HiLadder (˜500 kDA to 1,500 kDa) and Mega-A Ladder (1,500 kDa to 6,000 kDa) (from Hyalose) were run to determine the size of HA from each sample. Samples were run overnight at 4° C. at 75 volts with a 1 to 10 running ratio in TAE buffer. The gel was next stained as follows. The gel was placed in a 0.005% (w/v) Stains-All (Sigma-Aldrich) in 50% ethanol solution overnight. To de-stain, the gel was placed in ddH₂O for 48-hours and then placed under ambient light in ddH₂O for 4-hours to complete the final de-staining stages and then photographed under normal white light. The gel image is representative of five independent hyaluronic acid gel electrophoresis assays.

Hyaluronidase Cell Growth Assays.

NMRSF cells were seeded onto treated 6 cm polystyrene tissue culture plates (Corning) or soda lime glass slides (Nalge Nunc). Twenty-four hours post plating, the media was changed to either contain 3 U/mL hyaluronidase (HAse) from (Sigma) or no enzyme and changed every 48 hours with or without the enzyme. Images were taken on days 7 and 21 for +/−HAse using SPOT Advanced imaging software (Diagnostic Instruments). Reversion from complete confluence (CC) to early contact inhibition (E.C.I.) was obtained by incubating NMRSF cells with HAse for 12 days and then removing it for 4 days. Cell counts were taken for two independent experiments in triplicate on days 5, 10, 15 and 20 post seeding on a Beckman Coulter Z2 Particle Count and Size Analyzer. Similar methods were used for HSF cells. Day 20 conditioned media +/−HAse from NMRSF cells was added to HSF cells 24 hours post plating and changed every 48 hours. Cells were harvested, counted and fixed for cell cycle analysis on days 2, 3, 4 and 7. Cell cycle was analyzed using flow cytometry on a BD Biosciences FACSCanto flow cytometer to determine the % of cells in G1 phase.

### Results

Naked Mole Rat Cells Secrete High Molecular Weight Hyaluronic Acid.

We observed that while culturing adult NMR fibroblasts, the vacuum systems in our tissue culture hoods became clogged. We also noticed an increased viscosity of NMR cell culture media after it had been on the plate for several days. These observations were seen with mole rat fibroblasts and not with other rodent, human or NMR embryonic cells lines.

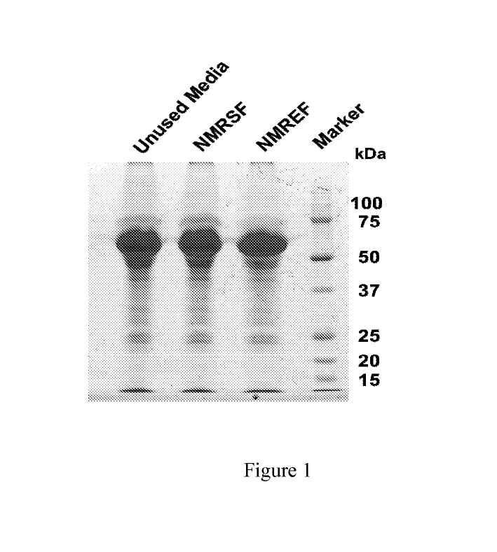

To test if the increased viscosity is due to a secreted protein, 150 μg of protein from unused complete EMEM media, Day 20 NMRSF and Day 20 NMREF conditioned media was run on a 4-15% SDS polyacrylamide gel and protein separation visualized by coomassie blue staining. No abnormalities in protein expression were detected from the increased viscous NMRSF media when compared to the non-viscous NMREF or unused media (FIG. 1). Thus, it does not appear that overexpression of a secreted protein(s) was the cause for increased viscosity. We also did not observe increased secretion of lipids as a factor for the change in viscosity, based on there being no hydrophobic/hydrophilic separations or fractions in the media, even after ultra-centrifuging the media at 70,000×g for 4-hours.

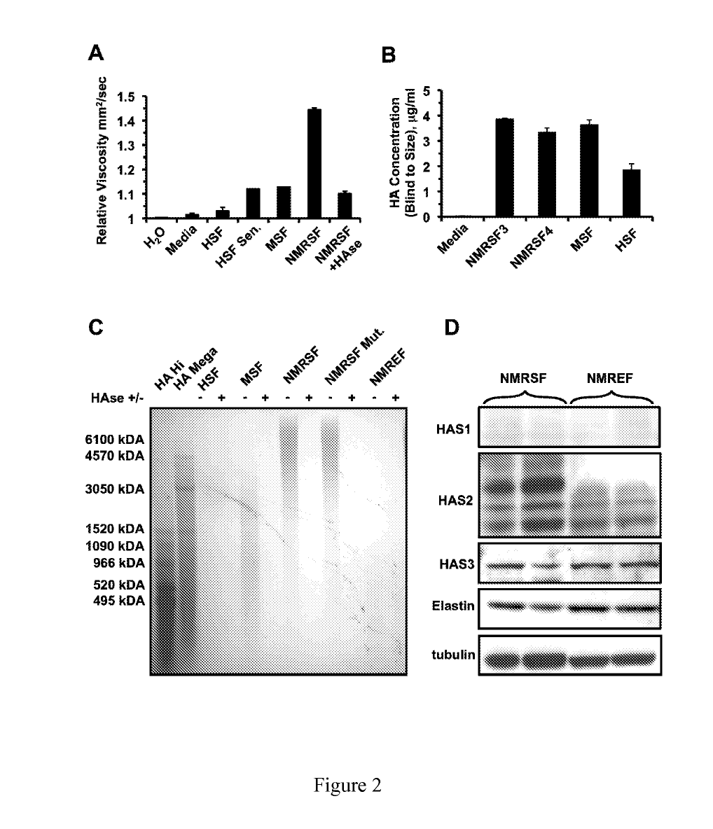

We tested the viscosities, relative to ddH₂O, using an Oswald viscometer of unused media, day 20 conditioned media from human skin fibroblasts (HSF), senescent human skin fibroblasts (HSF Sen.), mouse skin fibroblasts (MSF), naked mole rat skin fibroblasts (NMRSF), and NMRSF media that had been treated with hyaluronidase (HAse). HAse used was from Streptomyces hyalurolyticus (Sigma), which is specific for cleaving the β-D-GalNac-(1→4)-β-D-GlcA bond of HA and not other extracellular carbohydrates such as chondroitin and chondroitin sulfate. NMRSF media was 43% more viscous than fresh media, 41% more viscous than HSF media, 30% more viscous than HSF Sen media and 28% more viscous than MSF media (FIG. 2A). When treated with HAse, the viscosity of NMRSF media decreased by 32%, and was similar in viscosity to the other mammalian cells lines (FIG. 2A). Thus, HA was responsible for the increased viscosity of NMRSF media.

To determine if the overall concentration of HA molecules is different among NMRSF, MSF and HSF cell lines, we used an HA ELISA (FIG. 2B). To our surprise, there did not appear to be an increase in the overall amount of HA molecules present in NMRSF media compared to MSF media, while both of these cells lines appeared to have a slight increase in overall HA concentration; 95% compared to HSF media. This indicates that the increased viscosity of NMRSF media due to HA is not completely due to an overexpression or increase in HA molecules. It is important to note that the HA ELISA kit (Eschelon) determines HA concentration based on their uniform sized HA standards and does not take size of HA into account. HA can vary in size from just a few repeats of its disaccharide to over several mega Daltons and the HA-ELISA kit measures both as the same in terms of concentration. Thus, NMRSF cells do not make their media viscous by an overall overexpression and secretion of HA molecules, but possibly due to the size of HA that is produced.

To determine the size of HA in the media, we examined various conditioned media by agarose gel pulse field electrophoresis (FIG. 2C). By running commercially available HA size markers (Hylose), we were able to determine that NMRSF and NMFSR Mut. cell lines predominately contain HA of the high molecular weight (HMW) size of >6 MDa. Embryonic NMR fibroblast (NMREF) media, HSF media and MSF media contained HA of low molecular weight LMW-HA) in the range of <400 kDa to 3 MDa. Pre-treatment of the media with HAse removed all banding patterns/streaks on the gel, indicating that the patterns observed were due to HA. Thus, the increased viscosity of NMR fibroblast media is due to the secretion of HMW-HA.

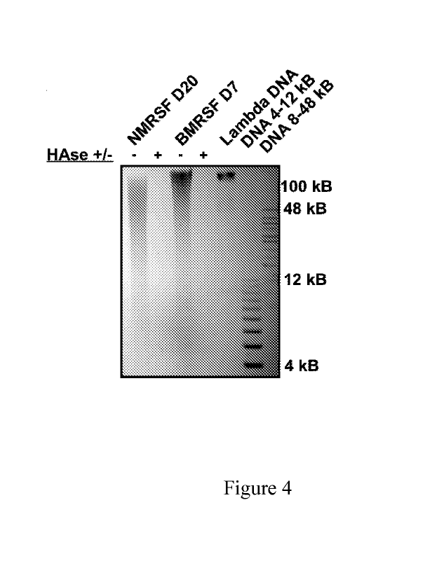

Similar to what was observed with NMRSF, we also observed that blind mole rat fibroblasts also produced high moleculear weight HA (FIG. 4). Even within 7 days of growth, blind mole rat cells appear to produce more and larger HMW-HA than NMRSF cells.

HA in multicellular organisms is synthesized by three different hyaluronic acid synthases (HAS) that are transmembrane proteins that enzymatically combine repeating units of D-glucuronic acid and N-acetyl-D-glucosamine linked by a β(1→4) bonds on the inside surface of the plasma membrane and export the sugar chain into the extra-cellular space. HAS1 and HAS2 are responsible for the production of high molecular weight HA, while HAS3 is responsible for the production of low molecular weight HA. We analyzed the protein expression of HAS1, HAS2, and HAS3 between HMW-HA expressing NMRSF cells and LMW-HA expressing NMREF cells by Western blot (FIG. 2D). HAS1 was only marginally detected in either cell line. HAS2, a highly conserved protein, with 99% identity in amino acid sequence between humans, mice and rats (ClustalW2 analysis), was overexpressed in NMRSF cells compared to NMREF cell lines. Also, multiple bands were detected for HAS2 in NMRSF, indicating possible posttranscriptional modifications that could affect the high production of such HMW-HA. There was no difference in HAS3 expression between NMRSF and NMREF. Also, we examined elastin expression as an additional means to determine if a protein is making NMRSF media viscous and not NMREF media. It again appears this is not the case, as no detectable differences in elastin protein expression were observed between NMRSF and NMREF cells.

These data suggest that NMRSF cells expressed very high molecular weight HA; >6 MDa, by the overexpression/upregulatoin of HAS2 and this leads to the increased viscosity of NMRSF media.

HMW-HA Promotes E.C.I. in NMRSF Cells.

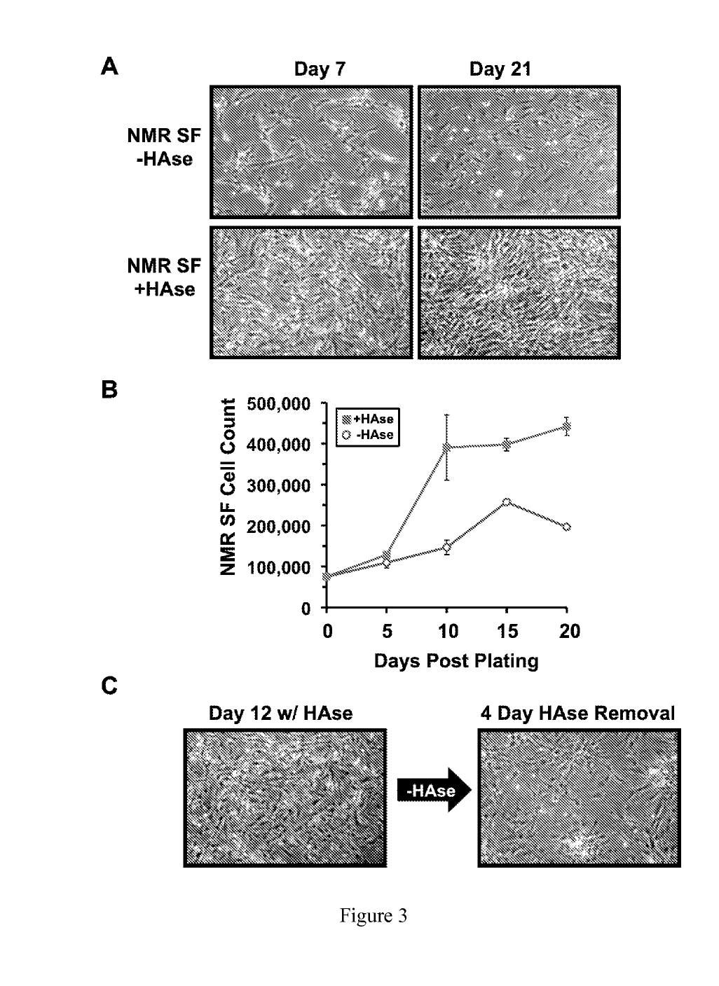

We decided to examine if the HMW-HA secreted by NMRSF goes beyond stimulating normal contact inhibition and acts as the extracellular signal for E.C.I. NMRSF cell cultures were grown for twenty-one days, with media removed and replaced with and without HAse every two days and images taken on days seven and twenty-one (FIG. 3A). NMRSF cells grown in their normal media without the addition of HAse displayed their stereotypical E.C.I. phenotype, while NMRSF cells treated with HAse obtained a visible increase in cell density by day seven, and by day twenty-one were completely confluent (C.C). This shows that by breaking down and removing both soluble and insoluble HA from the cell culture, we are able to stimulate NMR cells to grow to C.C. To determine if the presence of HAse simply stimulates rapid cell growth or actually acts to abolish ECI, we took a closer and more quantitative look at both early and late cell densities of NMRSF cell grown with and without HAse (FIG. 3B). At day five, when both cell cultures are still sparse, there is not a statistically significant difference in cell counts between HAse treated and non-treated plates. By day ten, however, there is a 2.7-fold (P=0.0067) increase in cell number on HAse treated plates. The difference between cell counts on HAse treated and non-treated plates remains significant at day 15; P=0.0001 and day 21; P=0.0001, and approximately 2-fold more after three weeks of growth. The ability to grow NMRSF cells to C.C. by treating the media for 12 days with HAse is lost when HAse containing media is removed and replaced with normal media, as the cells revert back to the E.C.I. phenotype (FIG. 3C). This reversion is accompanied by cell death and migration of the fibroblasts away from each other. In summary, the analysis of HMW-HA and its role on E.C.I. shows that HMW-HA secreted by NMRSF cells is required for the E.C.I. phenotype. This bypass of E.C.I. by the addition of HAse was not permanent, as removal of media containing HAse for several days resulted in the cells reverting back to E.C.I.

Tissues of NMRs Contain Increased Levels of HA.

We next examined HA in vivo Skin samples taken from NMR, mouse, and guinea pigs; which are phylogenetically the closest related “model research” rodent to NMRs, were fixed, treated with or without HAse and then stained for HA with alcian blue at pH 2.5. NMR skin samples show an increase in HA staining compared to mouse and guinea pig, and this staining is removed with the addition of HAse. This indicates that not only is there an abundance of HA produced in cell culture, but also in vivo, and this may play roles in NMR cancer resistance and longevity. These data also indicate that cells cultured from other tissues of mole rats could be used as a source of HMW HA.

Based on the increase in HA in skin tissues of NMRs compared to the other rodents, we examined HA staining by alcian blue in other tissues of the NMR and compared it to tissues from the guinea pig and from Eastern mole. The three tissues that had the greatest differential HA staining between NMRs and guinea pigs were the heart, brain and kidneys indicating cells from these animals lack the amounts of HA found in mole rat tissues. Treatment with HAse confirmed the alcian blue staining in NMRs was specific for HA, as the staining was not present after HAse treatment.

While specific embodiments have been presented in this description, those skilled in the art will recognize that routine modifications can be made by those skilled in the art without departing from the scope of the invention.

We claim:

1. A population of cells comprising fibroblasts obtained from a mole rat which fibroblasts have been cultured such that the fibroblasts produce hyaluronic acid having a molecular weight of at least 6,000 kDa.

2. The population of cells of claim 1, wherein the fibroblasts are skin fibroblasts.

3. The population of cells of claim 1, wherein the cells have been cultured for at least 100 days.

4. The population of cells of claim 1, wherein the cells have been stably transformed.

5. The population of cells of claim 1, wherein the cells have lost early contact inhibition and thereby can be grown to confluence.

6. The population of cells of claim 1, wherein the mole rat is naked mole rat.

7. A method for obtaining high molecule weight hyaluronic acid (HMWHA) producing cells comprising:
a) obtaining cells comprising fibroblasts from a mole rat;
b) culturing the cells for sufficient number of passages such that the cells produce HMWHA and do not exhibit early contact inhibition and.

8. The method of claim 7, wherein the mole rat is naked mole rat.

9. The method of claim 7, wherein the cells are passaged for at least 20 times.

10. A conditioned medium which is obtained by incubating the population of cells from claim 1 in a culture medium and allowed to condition for at least 2 hours.

11. The conditioned medium of claim 10, which is allowed to condition for 3 hours to 30 days.

12. The conditioned medium of claim 10, which is free of cells or cellular debris.

13. The conditioned medium of claim 7, which comprises at least 50 ng of HMWHA per ml of the conditioned medium.

14. The conditioned medium of claim 13, which comprises from 1 to 5 μg HMWHA per ml of the conditioned medium.

15. The conditioned medium of claim 10, further comprising a pharmaceutical carrier.
